(19) United States
(12) Patent Application Publication
(10) Pub. No.: US 2012/0191074 A1
(43) Pub. Date: Jul. 26, 2012

(54) REDUCED SIZED PROGRAMMABLE PUMP

(75) Inventor: Bernd Steinbach, Friedberg (DE)

(73) Assignee: PALYON MEDICAL (BVI) LIMITED, Tortola (VG)

(21) Appl. No.: 13/011,053

(22) Filed: Jan. 21, 2011

(51) Int. Cl. A61M 37/00

(52) U.S. Cl. 604/891.1

(57) ABSTRACT

A reduced size implantable infusion pump is disclosed. The pump preferably includes a lower profile pump housing facilitated by specific propellant envelope configurations. For instance, the pump may include one or more c-shaped propellant envelopes that each define active substance and propellant chambers.

### BACKGROUND OF THE INVENTION

The present invention relates to implantable pumps, more particularly, a reduced size implantable pump capable of varying flow rates of active substances from the pump to the patient.

Implantable pumps have been well known and widely utilized for many years. Typically, pumps of this type are implanted into patients who require the delivery of medication or other fluids (hereinafter referred to as “active substances”) to specific areas of their body. For example, patients that are experiencing severe pain may require pain killers daily or multiple times per day. Absent the use of an implantable pump or the like, a patient of this type would be subjected to one or more painful injections of such active substances. In the case of pain associated with more remote areas of the body, such as the spine, these injections may be extremely difficult to administer and particularly painful for the patient. Furthermore, attempting to treat conditions such as this through oral or intravascular administration of an active substance often requires higher doses and may cause severe side effects. Therefore, it is widely recognized that utilizing the implantable pump may be beneficial to both the patient and the treating physician.

Many implantable pump designs have been proposed. For example, U.S. Pat. No. 4,969,873 (“the '873 Patent”), the disclosure of which is hereby incorporated by reference herein, teaches one such design. The '873 Patent is an example of a constant flow pump, which typically includes a housing having two chambers, a first chamber for holding the active substance to be administered to the patient and a second chamber for holding a propellant. A flexible membrane separates the two chambers such that expansion of the propellant in the second chamber pushes the active substance out of the first chamber. This type of pump also typically includes an outlet opening connected to both the first chamber and a catheter or other delivery device for directing the active substance to the desired area of the body, a replenishment opening for allowing refilling of the first chamber, and bolus opening for allowing the direct introduction of an active substance through the catheter without introduction into the first chamber. The replenishment and bolus openings are typically each covered by septa to allow a needle or similar device to be passed therethrough, but that reseals upon removal of same. As pumps of this type provide a constant flow of active substance to a specific of the body, they must be refilled periodically with a proper concentration of active substance suited for extended release.

Implantable pumps may also be of the programmable type, meaning that they can provide variable flow rates of an active substance therefrom. While these types of programmable pumps have typically involved the use of a solenoid pump or peristaltic pump, as opposed to the above-discussed constant flow-type pumps, certain pumps similar to the above-discussed constant flow pumps have been modified in order to provide the ability of varying flow rates of an active substance therefrom. For instance, U.S. Patent Application Publication Nos. 2007/0005044 and U.S. Pat. No. 7,637,892 (“the '892 Patent”), the disclosures of which are hereby incorporated by reference herein, teach such pumps. Programmable pumps are indeed important for allowing a medical professional, or even the patient, to vary the flow of an active substance from the pump. Obviously, there may be times that require more or less medication to be dispensed from the pump. This could also be something dictated by the environment to which the patient is subjected.

Of course, any time an instrument is meant for implantation in the human body, the overall size of such device is of concern. Depending upon the implantation site of any of the above-discussed implantable pumps, the overall size of such device may impact both the comfort and appearance of the patient. For instance, with some large implantable pumps, implantation may be impossible or very uncomfortable for the patient, and/or may result in noticeable bulges in the patient's body. Therefore, there exists a need for a reduced size implantable pump that is capable of varying flow rates of active substances from the pump to the patient.

### BRIEF SUMMARY OF THE INVENTION

A first aspect of the present invention is a reduced size implantable pump for dispensing an active substance at one or varying flow rates to a patient. In accordance with one embodiment of the first aspect, the pump includes a pump housing defining an interior and having a top surface and a bottom surface; a valve disposed within the interior, the valve being capable of moving in a direction extending between the top and bottom surfaces; a propellant envelope disposed within the interior, the propellant envelope defining an active substance chamber and a propellant chamber; and a hermetic housing attached to the pump housing, the hermetic housing including a first pressure sensor, a second pressure sensor, and an actuator capable of moving the valve.

In accordance with other embodiments of this first aspect, the pump housing may include a middle part between top and bottom parts. The valve may be disposed within an aperture formed in the middle part. The hermetic housing may be disposed between the bottom and middle parts. The pump may further include a catheter fluidly connected with the active substance chamber, and/or a resistor capillary fluidly connected with the active substance chamber.

In accordance with certain embodiments of this first aspect, the first pressure sensor measures the pressure of fluid within the active substance chamber, and fluid dispelled from the active substance chamber flows through the resistor capillary, into contact with the second pressure sensor, through the valve, and through the catheter. The pressure information obtained by the first and second pressure sensors is used to determine whether the actuator should actuate the valve. To accomplish this, the hermetic housing further includes a circuit board, motor and battery.

Further, the pump of the first aspect may include first and second propellant envelopes, where the first and second propellant envelopes each include first and second membranes and a propellant that expands under normal body temperature to act upon the flexible membranes. In certain embodiments, the propellant envelope is substantially c-shaped. The pump may also include a replenishment port at least partially surrounded by the propellant envelope.

Another aspect of the present invention is another reduced size implantable pump for dispensing an active substance at one or varying flow rates to a patient. In accordance with one embodiment of this second aspect, the pump includes a pump housing defining an interior and having a top surface and a bottom surface; a valve disposed within the interior, the valve being capable of moving in a direction extending between the top and bottom surfaces; a first propellant envelope disposed within the interior, the first propellant envelope being c-shaped; a second propellant envelope disposed within the interior, the second propellant envelope being c-shaped.

In other embodiments of this second aspect, the pump further includes a hermetic housing attached to the pump housing, the hermetic housing including a first pressure sensor, a second pressure sensor, and an actuator capable of moving the valve. The pump may also include a middle part between top and bottom parts, the first propellant envelope and a top surface of the middle part defining a first active substance chamber, and the second propellant envelope and a bottom surface of the middle part defining a second active substance chamber. The valve may be disposed within an aperture formed in the middle part. The hermetic housing may be disposed between the bottom and middle parts. The pump may also include a catheter fluidly connected with the first and second active substance chambers, as well as a resistor capillary fluidly connected with the first and second active substance chambers. Still further, the hermetic housing may further include a circuit board, motor and battery. The first and second propellant envelopes may each include first and second membranes separating the active substance and propellant chambers, and the pump may include a replenishment port at least partially surrounded by the propellant envelope.

### BRIEF DESCRIPTION OF THE DRAWINGS

A more complete appreciation of the subject matter of the present invention and the various advantages thereof, can be realized by reference to the following detailed description in which reference is made to the accompanying drawings in which:

FIG. 1 is a top perspective view of a reduced size implantable pump in accordance with an embodiment of the present invention.

FIG. 2 is a side view of the implantable pump shown in FIG. 1.

FIG. 3 is a top view of the implantable pump shown in FIG. 1.

FIG. 4 is a top perspective view of a top part of the implantable pump shown in FIG. 1.

FIG. 5 is a bottom perspective view of the top part shown in FIG. 4.

FIG. 6 is a top perspective view of a middle part of the implantable pump shown in FIG. 1.

FIG. 7 is a bottom perspective view of the middle part shown in FIG. 6.

FIG. 8 is a top perspective view of a bottom part of the implantable pump shown in FIG. 1.

FIGS. 9a-b are a cross-sectional top view of the implantable pump shown in FIG. 1 taken along the line A-A in FIG. 2 and cross-sectional perspective view of the middle part with other components contained therein.

FIGS. 10a-c are a top view, first cross-sectional view, and second cross-sectional view of a propellant envelope for use in the implantable pump shown in FIG. 1.

FIG. 11 is a cross-sectional side view of the implantable pump shown in FIG. 1 taken along the line B-B in FIG. 3.

FIG. 12 is a cross-sectional side view of the implantable pump shown in FIG. 1 taken along the line C-C in FIG. 3.

FIG. 13 is a cross-sectional side view of the implantable pump shown in FIG. 1 taken along the line D-D in FIG. 3.

FIG. 14 is a top perspective view of a hermetic unit for use in the implantable pump shown in FIG. 1.

FIG. 15 is a bottom perspective view of the hermetic unit shown in FIG. 15.

FIG. 16 is a top cross-sectional view of the hermetic unit shown in FIGS. 14 and 15 with a bottom portion removed therefrom.

### DETAILED DESCRIPTION

In describing the preferred embodiments of the subject matter illustrated and to be described with respect to the drawings, specific terminology will be used for the sake of clarity. However, the invention is not intended to be limited to any specific terms used herein, and it is to be understood that each specific terms used herein, and it is to be understood that each specific term includes all technical equivalents which operate in a similar manner to accomplish a similar purpose.

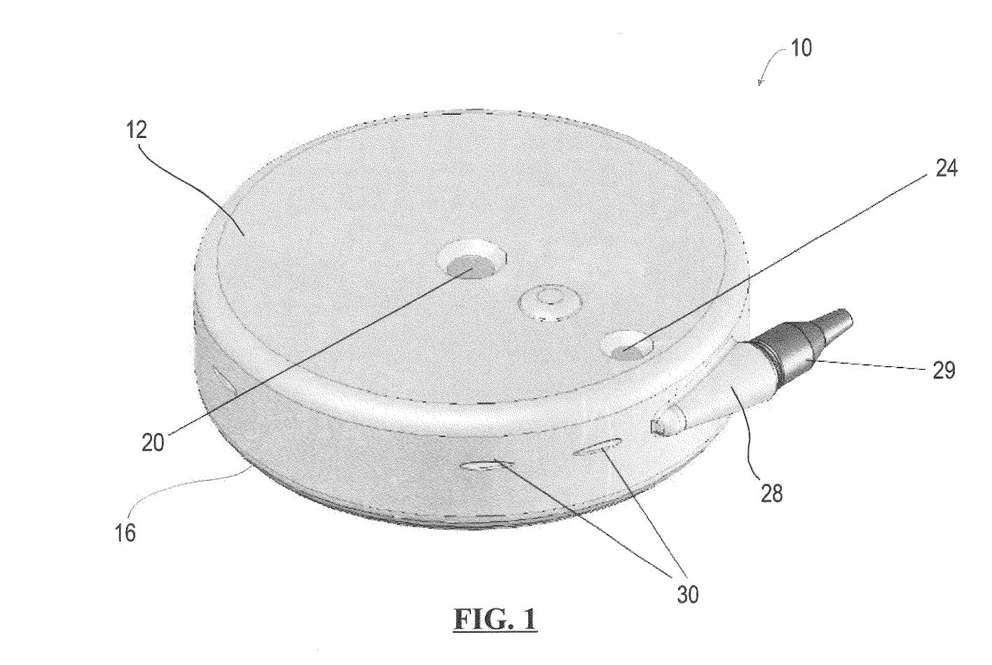

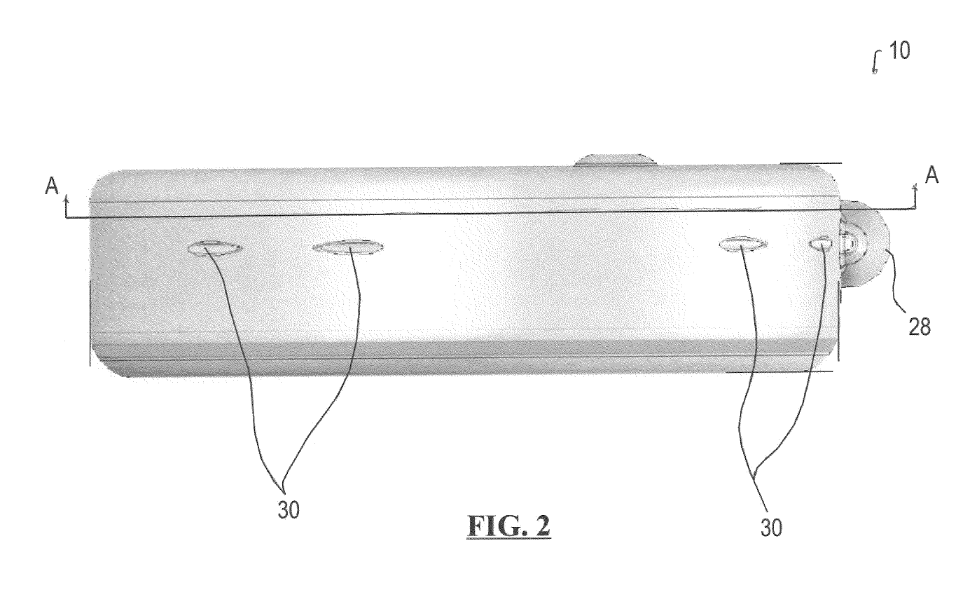

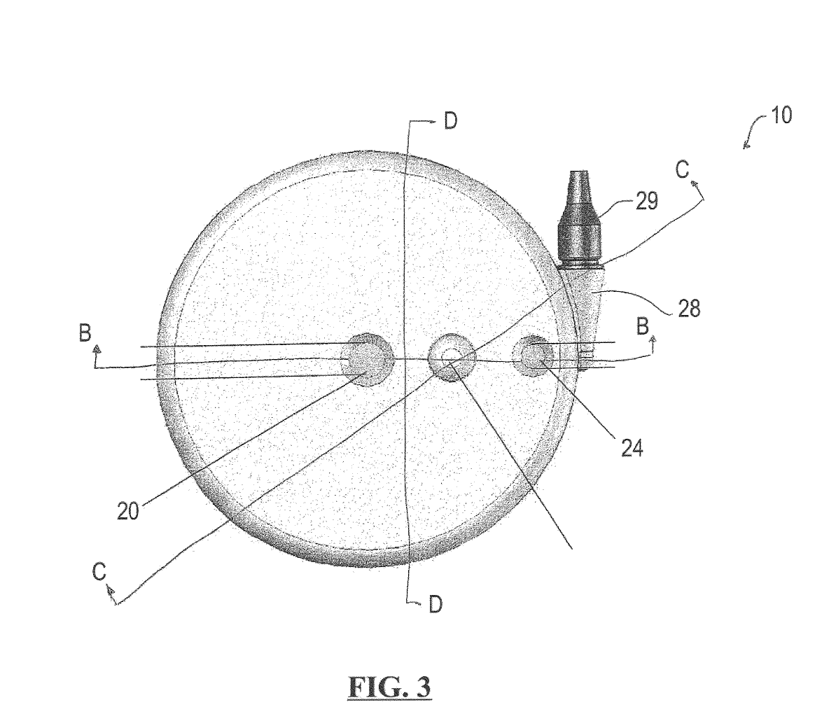

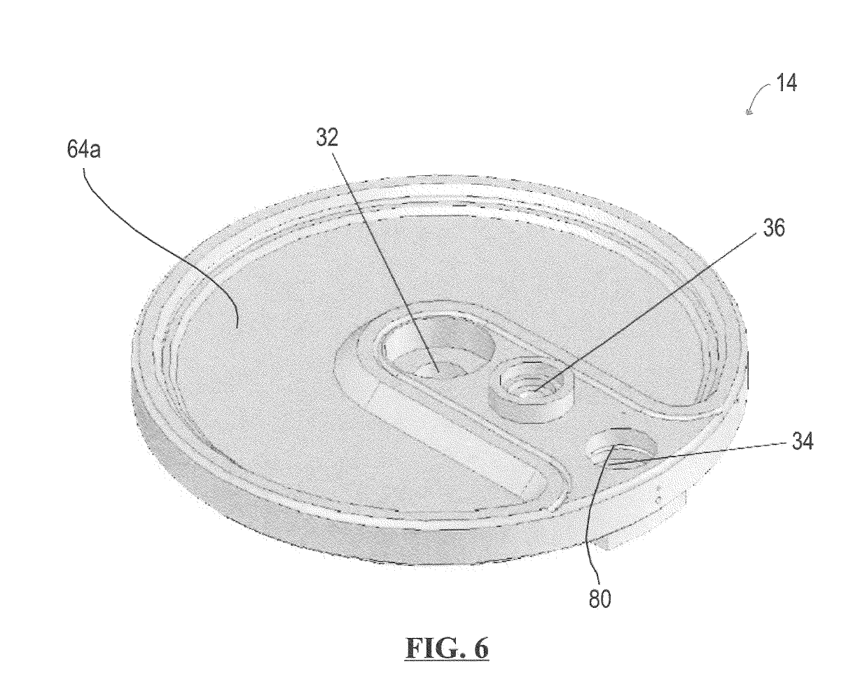

Referring to the drawings wherein like reference numerals refer to like elements, there is shown in FIGS. 1-3, in accordance with an embodiment of the present invention, a reduced sized programmable implantable pump designated generally by reference numeral 10. Pump 10 includes top part 12, middle part 14 (best shown in FIGS. 6, 7, 8 and 11-13) and bottom part 16. As is more fully described below, those three parts are mechanically attached to one another so as to contain the remaining components of pump 10 in an assembled construction. Top, middle, and bottom parts 12, 14, and 16 are, in the preferred embodiment, constructed of polyetheretherketone (“PEEK”). However, it is contemplated to construct such components out of differing materials, such as other like polymers or even metallic materials, like stainless steel or titanium. Moreover, in the illustrated preferred embodiment, the three parts of pump 10 are shown as being of a circular shape, but it is to be understood that the three parts may be of any shape suitable for implantation in the human body, the only constraint being that the three parts be of shapes suitable for cooperation with each other and the other remaining components of the pump. It is desirable to design pump 10 so that it does not contain any sharp edges or the like, as such could lead to problems during or after implantation of the pump.

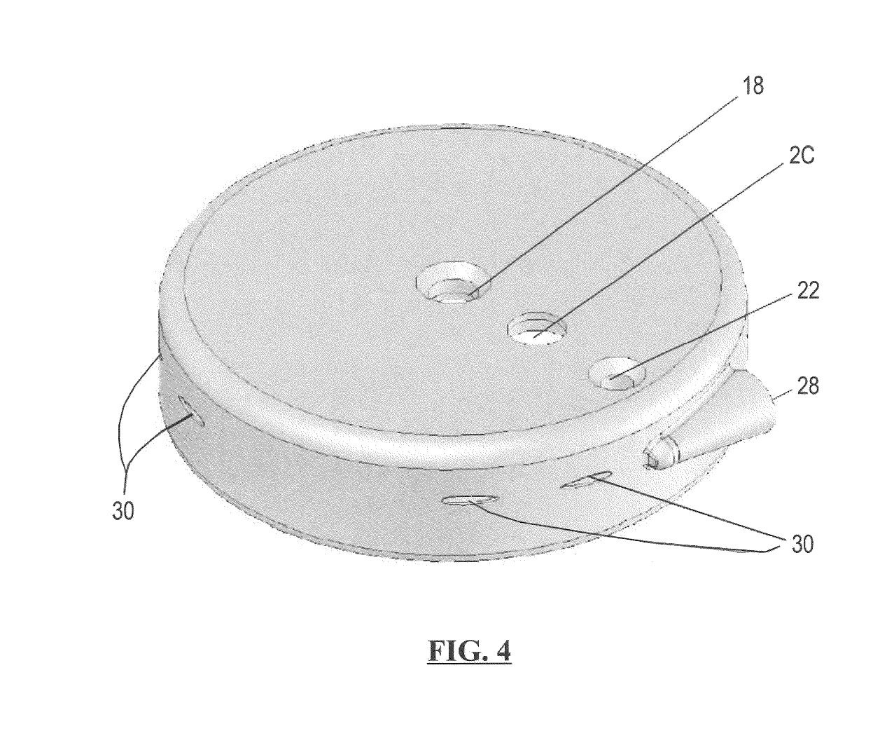

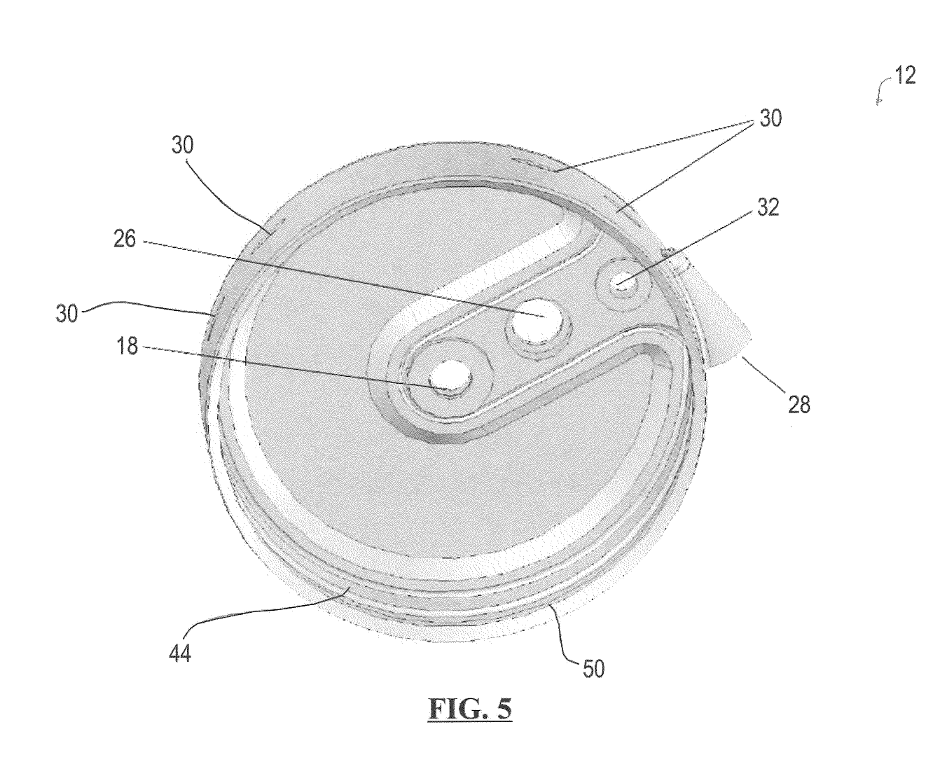

As shown, top part 12 includes an aperture 18 (best shown in FIGS. 4 and 5) that allows access to a central port useful in refilling pump 10. In a fully constructed state, such as shown in FIG. 1, a septum 20 overlies the central port. Top part 12 also includes a similar aperture 22 (also best seen in FIGS. 4 and 5) that allows access to a port that allows for direct access to the catheter, so that fluid injected through this direct access port goes directly to the catheter without passing through other portions of the pump. Again, in a fully constructed state, such as shown in FIG. 1, a septum 24 overlies the direct access port. Aperture 26 is also formed through top part 12, and, as will be discussed more fully below, is provided in order to allow for proper operation of a valve useful in varying flow rate of an active substance from the pump. Finally, top part 12 includes a catheter connector housing 28 for receiving a catheter connector 29 and various suture holes 30 for receiving sutures to when affixing pump 10 within the body of a patient.

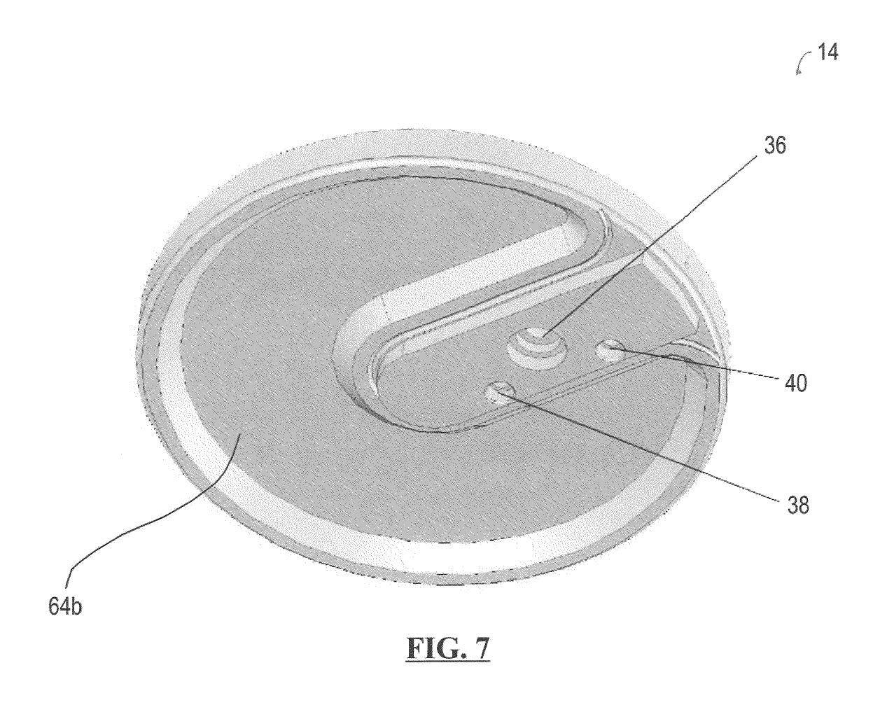

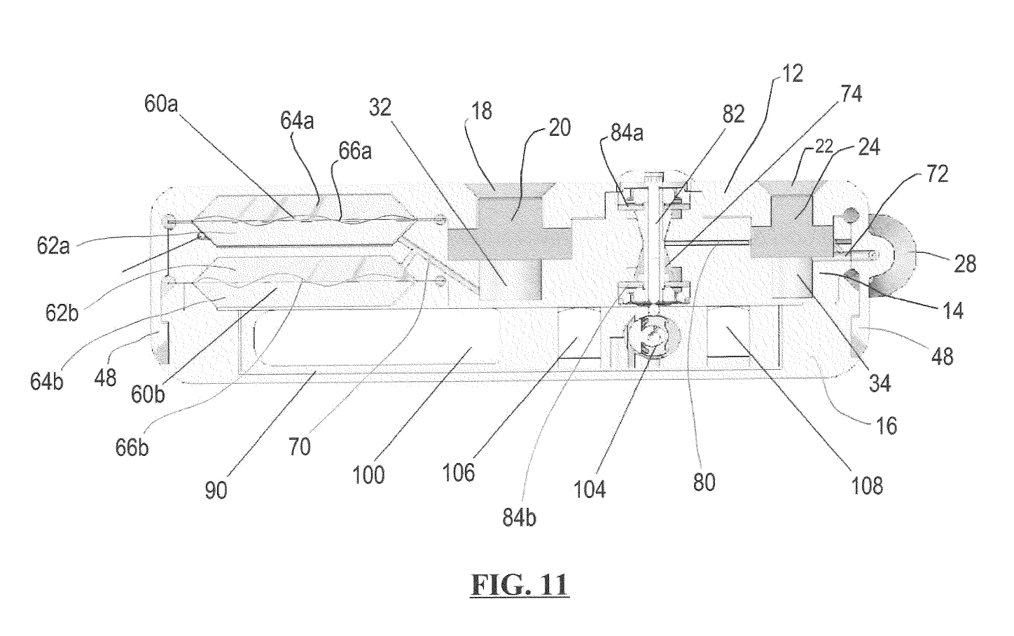

Pump 10 also includes a middle part 14, which is best shown in FIGS. 6 and 7. Middle part 14 includes an aperture 32 that partially defines the central port, and an aperture 34 that partially defines the direct access port. In the embodiment shown, neither of apertures 32 and 34 are completely formed through middle part 14, but rather, such apertures are in communication with certain ducts that are shown and will be discussed below in the discussion pertaining to FIG. 11. Middle part 14 also includes a valve housing 36, and, as is best shown in the view of FIG. 7, a lower surface of middle part 14 includes apertures 38 and 40 for receiving portions of first and second sensors, respectively. These additional elements will be discussed more fully below.

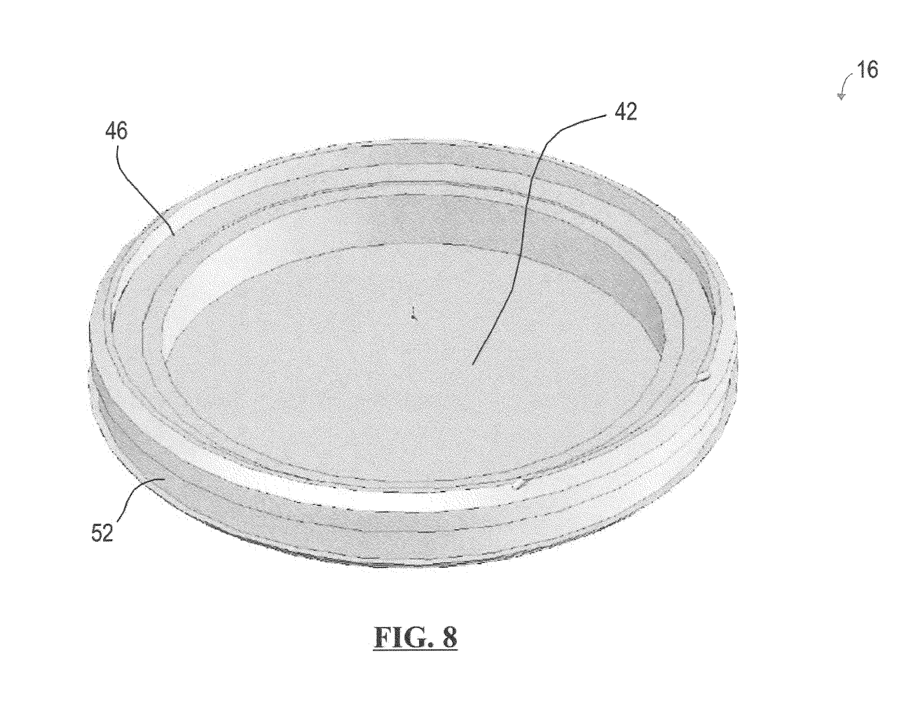

Pump 10 also includes, as is highlighted in FIG. 8, bottom part 16, which is designed to receive a hermetic unit (discussed below) within its circular chamber 42. In the fully constructed preferred embodiment shown in, for instance, FIGS. 1-3, middle part 14 is designed to be sandwiched between top part 12 and bottom part 16. This interrelationship among the three parts can best be seen in the cross-sectional views of FIGS. 11-13. Essentially, middle part 14 is received within a groove 44 formed in the underside of top part 12 (best shown in FIG. 5), as well as in a similar groove 46 formed on the top side of bottom part 16 (best shown in FIG. 8). With specific reference to FIGS. 11-13, top part 12 and bottom part 16 are designed to snap fit together at connection 48 thereby holding the three parts (as well as other components) of pump 10 together. As shown, top part 12 is formed at its underside with a male portion 50 that extends around its periphery (best shown in FIG. 5) that is designed for cooperation with a female portion or shoulder 52 that is formed around the periphery of bottom part 16 (best shown in FIG. 8). While a snap-fit connection is shown in a preferred embodiment illustrated in the drawings, any other suitable connection may be employed, including screwable connections or the like.

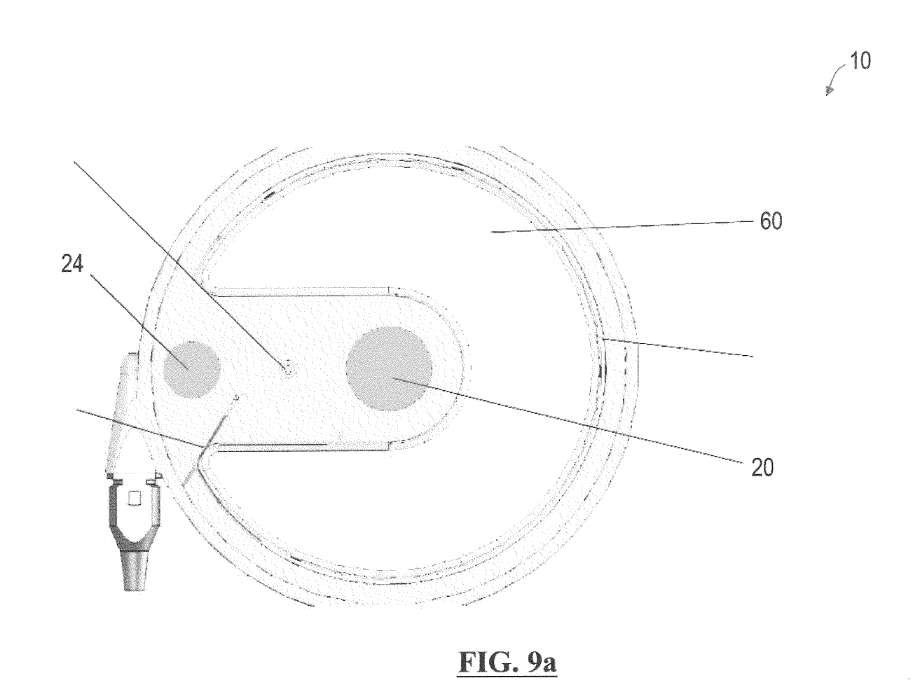

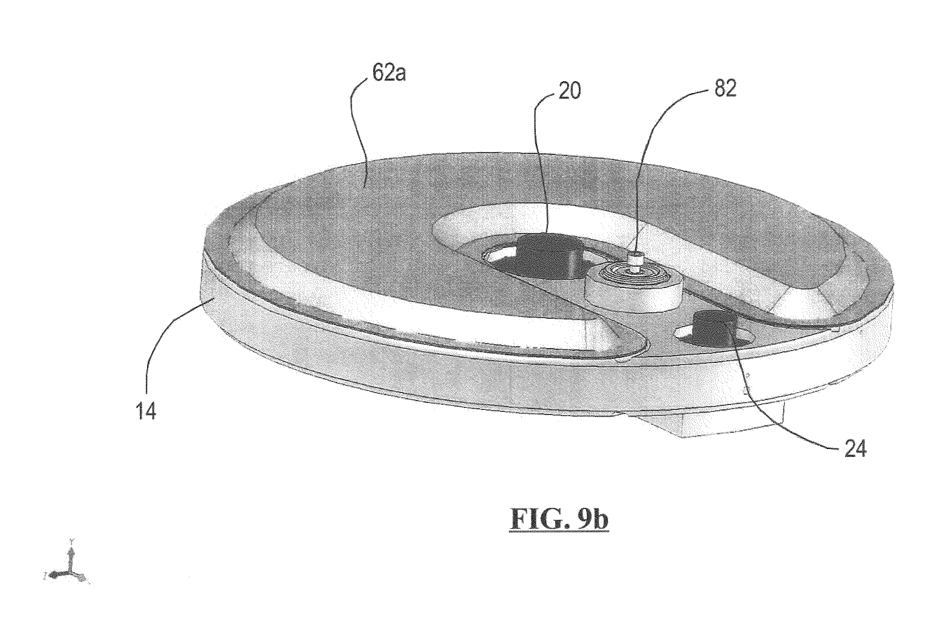

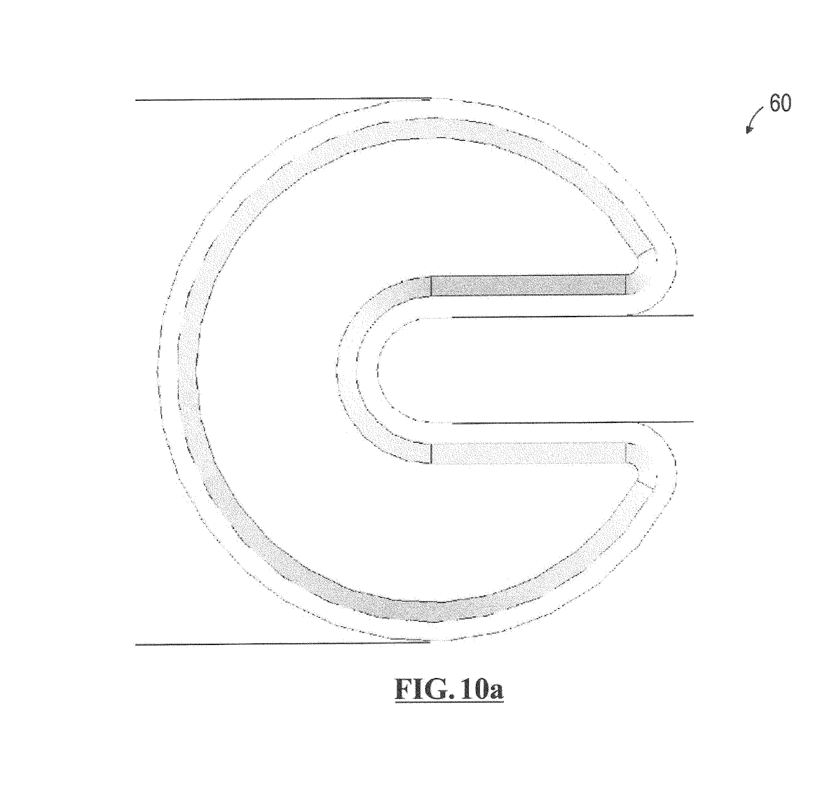

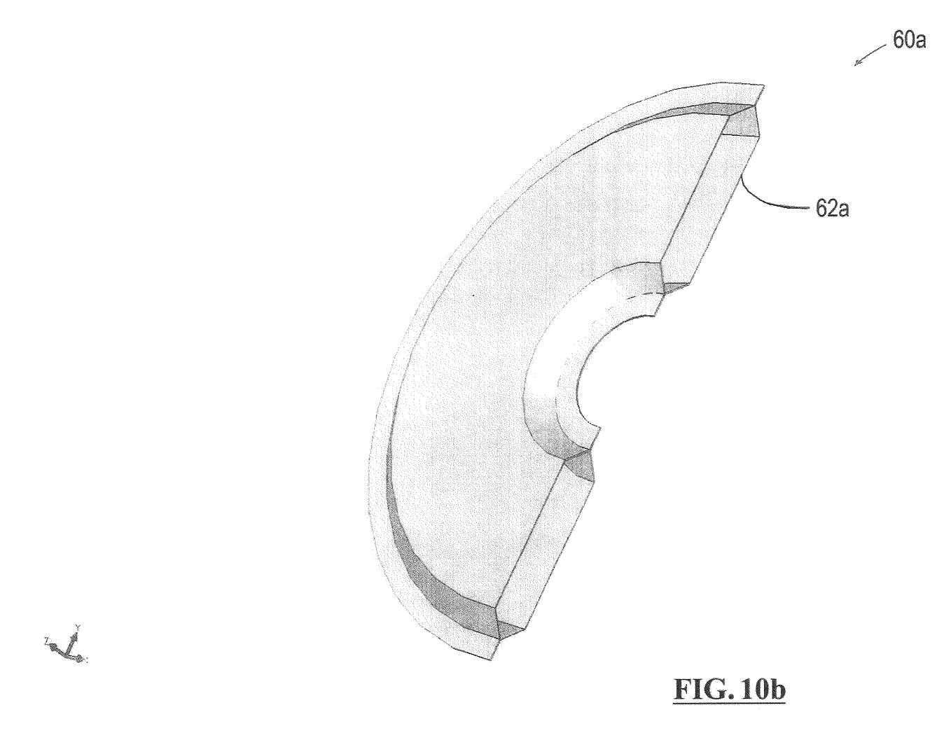

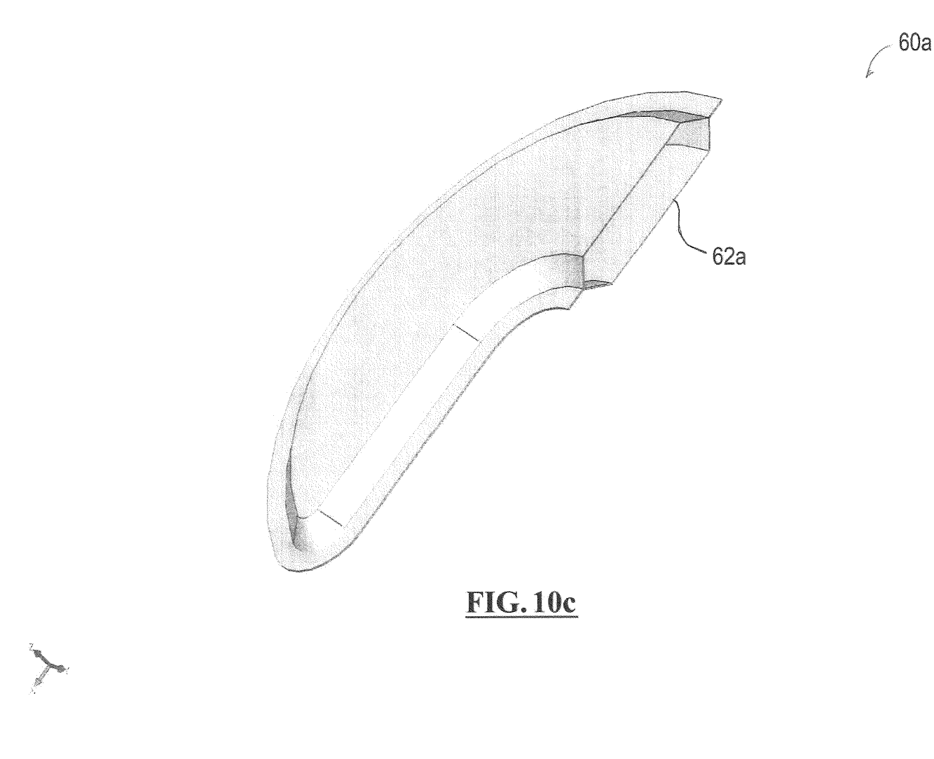

Now that the outer casing of pump 10 has been described, the present application will now focus on the internal components of same. With reference to the cross-sectional top view of FIG. 9a and cross-sectional perspective view of FIG. 9b, it is shown that pump 10 also includes at least one propellant envelope or chamber element 60. Propellant envelope 60, which is shown alone in FIGS. 10a-c, is designed so as to define an enclosed chamber 64 for housing a propellant (best shown in FIG. 11). Propellant envelope 60 may be constructed of any suitable material, including flexible materials. In a preferred embodiment, propellant envelope 60 is constructed of identical pieces of deep drawn aluminum laminate fills sealed at their rims. Moreover, propellant chamber 64 may be defined not only by a lower portion of propellant envelope 60 and flexible membrane 66, but may also include a secondary flexible membrane that creates an enclosed flexible propellant chamber. The propellant envelope is shown in FIGS. 9 and 10 as being C-shaped. In other words, a large majority of element 60 is circular shaped so as to extend around a portion of pump 10, but a portion is cutout in order to provide room for the central port, the valve (discussed below), and the catheter direct access port. This, along with other aspects of pump 10, allows the overall height of the pump to be reduced from that of prior art designs. Of course, although a C-shape is shown, other shapes may be employed to achieve the same purpose. For instance, it is contemplated that element 60 may be of an “O” or donut shape, a J-shape, a D-shape, or even partial variations thereof. For instance, it is contemplated that element 60 could be only extend around one half of pump 10.

In the preferred embodiment shown in the drawings, pump 10 includes two propellant envelopes 60a and 60b, which each cooperate with other portions of pump 10 in order to define an active substance chamber. The two elements are best depicted in FIGS. 11-13, where like elements between the two propellant envelopes are identified with either an ‘a’ or ‘b’ identifier. In the preferred embodiment shown, the propellant envelopes are oriented in an up-side-down manner with respect to one another. Specifically, envelope 60a is situated so that its lower membrane 62a cooperates with a surface 64a of the top side middle part 14, where envelope 60b is situated so that its upper membrane 62b cooperates with a surface 64b of the bottom side of middle part 14. It is to be understood that during operation, only membranes 62a and 62b of elements 60a and 60b move. More particularly, during filling, those membranes moves towards the other membrane of the particular envelope, while during dispensing (i.e., when the propellant contained between the membranes expands), those membranes move away from the other membrane of the particular envelop. As also shown in FIGS. 11-13, the central port (specifically aperture 32) is connected to each of propellant envelopes 60a and 60b by bifurcated duct 70. Likewise, the catheter direct access port (specifically aperture 34) is connected to catheter connector housing 28 via a duct 72. Both ducts 70 and 72 are formed within middle part 14.

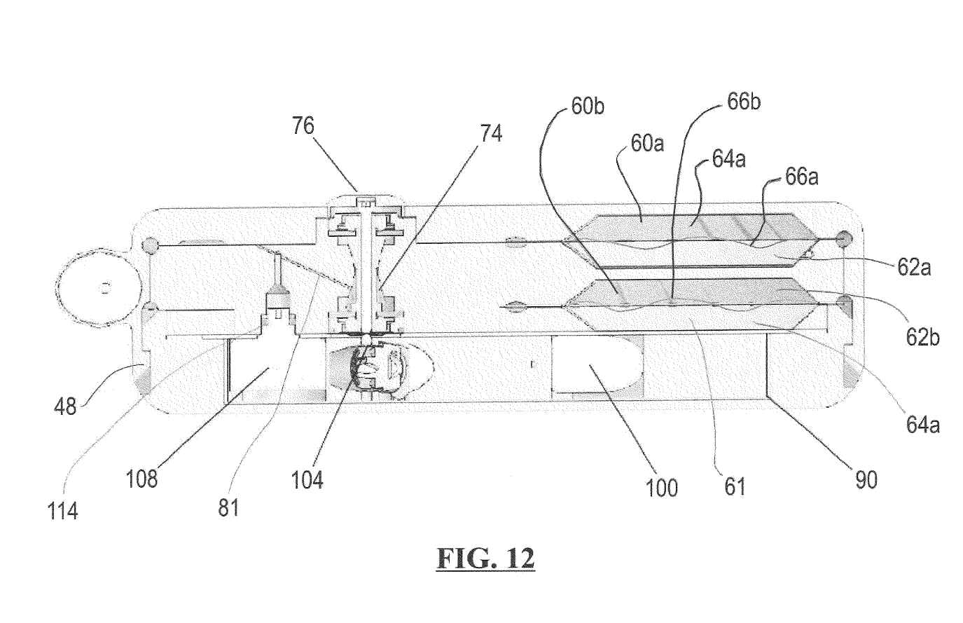

The different cross-sectional views of FIGS. 11-13 also depict a valve 74 contained within valve housing 36 of middle part 14 and a valve cover 76 covering aperture 26 of top part 12. Fluid dispelled from active substance chambers 62a and 62b is fed to valve 74 via a filter 78a and capillary 78b, as is known in the art and best shown in FIG. 9a. Essentially, as is also best shown in FIG. 11, fluid dispelled from the upper chamber passes through bifurcated duct 70. These fluids are then capable of intermingling, as well as entering through filter 78a and into capillary 78b. From the capillary, the fluid can travel through a duct 81 (best shown in FIG. 12) to valve 74. After passing through valve 74, the fluid can pass to catheter connector housing 28 via duct 80 (best shown in FIG. 11, but also shown in FIG. 6). Valve 74 is of a varying cross section, so that depending upon its positioning within valve housing 36, the flow rate of fluid flowing therethrough may be varied. In addition, valve 74 includes a valve adjustment 82, which is essentially an adjustment screw for changing the height of the valve to fine tune operation thereof. Essentially, valve 74 may be biased within housing 36 via silicone disks 84a and 84b, or the like. This biasing forces the valve into an initial position, where it will remain absent a force being applied thereto. The vertical orientation of valve 74 allows for middle part 14 to house the valve (via housing 36) without the need for a separate valve housing component. Moreover, the vertical orientation allows for valve 74, the central port, and the catheter direct access port to be disposed in what is essentially open space provided by the shape of propellant envelopes 60a and 60b. All of this aids in the reduction in size of pump 10.

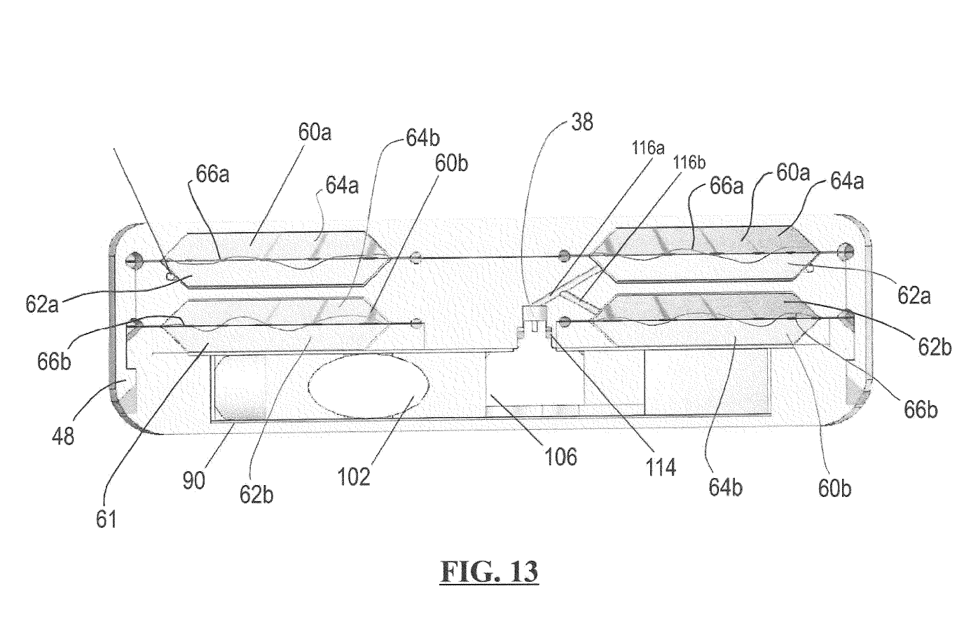

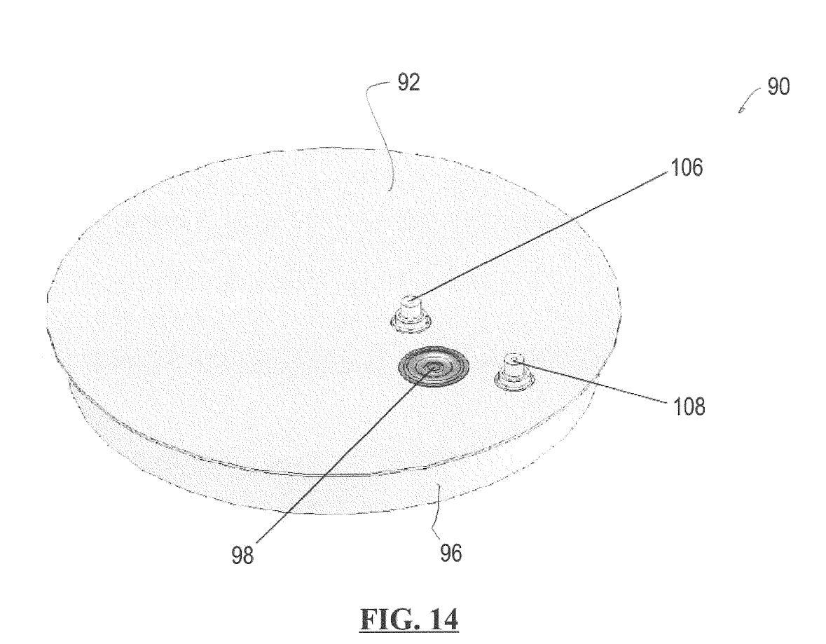

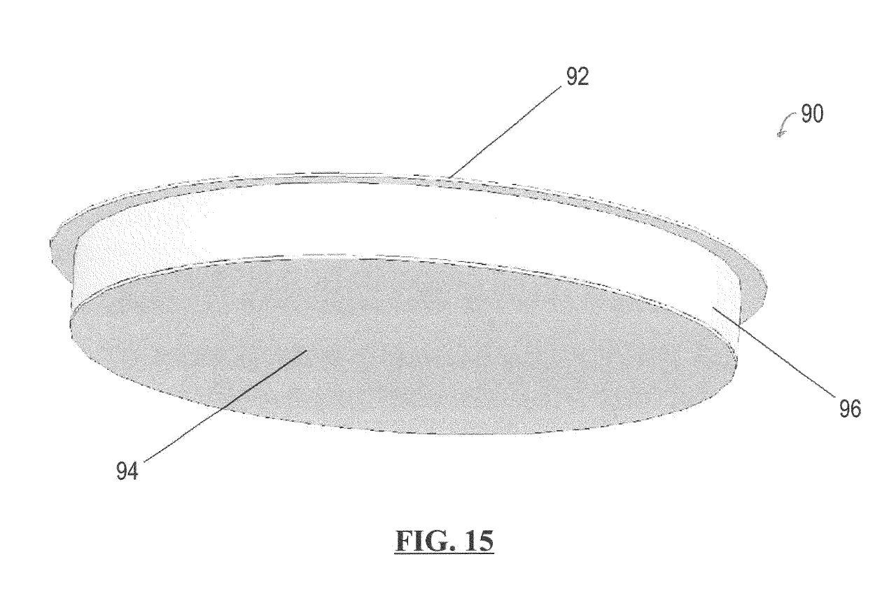

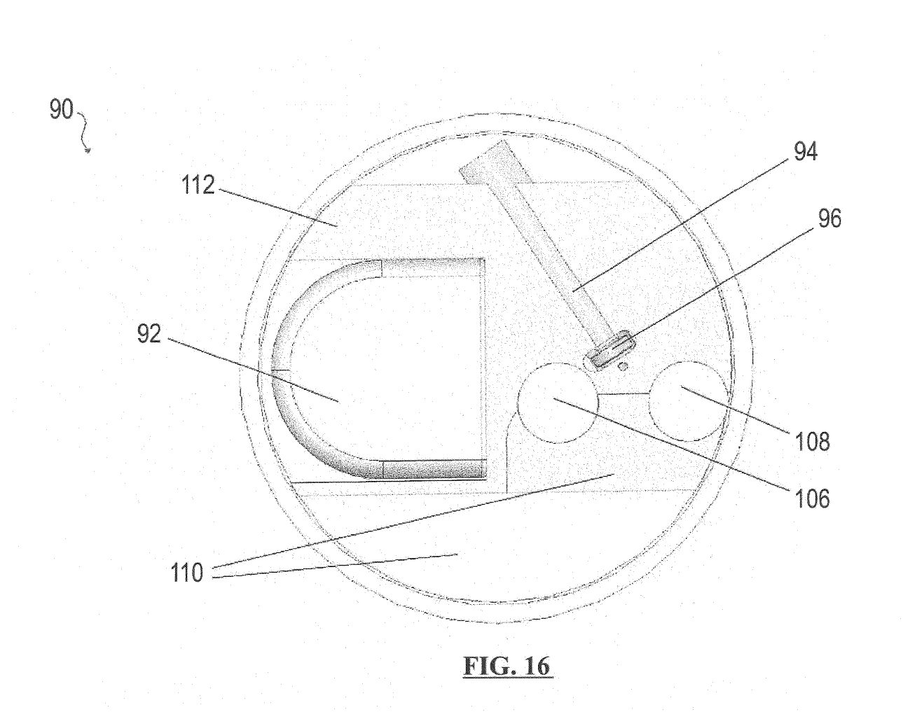

To this point, the above-discussion has largely focused on the components contained within either top part 12 or middle part 14. However, as noted above, bottom part 16 is designed to house a hermetic unit 90, which can be seen alone in FIGS. 14-16. Hermetic unit 90 is preferably constructed so as to completely seal the components it houses from the remainder of pump 10, including any fluid associated with the pump or the body in which pump 10 is implanted. In the embodiment shown, hermetic unit is largely constructed of titanium, preferably in a three-piece manner. Specifically, with reference to FIG. 15, hermetic unit 90 preferably includes a top plate 92, a bottom plate 94, and a ring wall 96. Top plate 92 preferably includes three apertures formed therethrough (best shown in FIG. 14), one of which is covered by a membrane 98 and two of which allow for portions of two sensors to extend therethrough (discussed below). Contained within hermetic unit 90 are several components shown throughout FIGS. 11-13 and 16, including a battery 100, motor 102 with eccentric cam 104, a first pressure sensor 106, a second pressure sensor 108, and a circuit board 110 that contains a microprocessor or the like. The various components housed within hermetic unit 90 are preferably held in place via a component support 112 (best shown in FIG. 16). As noted above and best shown in FIG. 16, a portion of first pressure sensor 106 and a portion of second pressure sensor 108 extend through apertures formed in top plate 92. The portion of each pressure sensor 106 and 108 that extends through the aperture is preferably surrounded by an o-ring 114 to ensure that the portions of the sensors that extend into middle part 14 are sealed. The o-ring surrounding first pressure sensor 106 is shown in FIG. 13, where it is also shown how the portion that extends through top plate 92 also extends into aperture 38 of middle part 14. The o-ring surrounding second pressure sensor 108 is shown in FIG. 12, where it is also shown how the portion that extends through top plate 92 also extends into aperture 40 of middle part 14.

In operation, pump 10 works in a similar fashion to pump 800 of '892 Patent, which has been incorporated by reference above. Propellants contained within propellant chambers 64a and 64b preferably exert a force upon membranes 66a and 66b to thereby force active substance contained within active substance chambers 62a and 62b therefrom. As in the prior art, the propellants (which may be the same propellant) preferably expand isobarically under normal body heat. The expelled active substance (which may be the same in each of chambers 62a and 62b) travel both towards first pressure sensor 106 via ducts 116a and 116b (best shown in FIG. 13). The sensor preferably takes a pressure reading of the active substance, which can be transmitted to the microprocessor located on circuit board 110. This pressure reading taken by first pressure sensor 106 essentially denotes the pressure from within active substance chambers 62a and 62b. Fluid is also dispelled towards valve 74, as described above. In addition, a second pressure is read by second pressure sensor 108. This second pressure reading is dependent upon the positioning of valve 74 downstream of the sensor. The two different pressure readings are preferably fed to the microprocessor, which can preferably utilize the pressure readings to calculate the flow rate of fluid being expelled from pump 10. If the calculated flow rate of the active substance is not desired, the microprocessor located on the circuit board can also trigger motor 102 to actuate cam 104. Since the cam abuts membrane 98, which abuts valve adjustment 82, the actuation can cause valve 74 to move within housing 36. This, in turn, changes the flow rate of active substance passing through the housing, as well as the pressure reading being taken by sensor 108.

Hence, pump 10 allows for a constant varying of flow rate of active substance being expelled therefrom. It is to be understood that while not shown in any of the figures, pump 10 may also include an antenna or other communications means for receiving input from a medical professional or the patient to set the desired flow rate. Likewise, it is contemplated to have pump 10 compatible with a handheld device or the like for setting the desired flow rate, as well as performing other functions with respect to the pump. For instance, the handheld device may provide a real time reading of the amount of medication remaining in the pump or even the fact that active substance is being re-introduced into the active substance chambers during a refill process. Moreover, the handheld device could be utilized to cease all fluid flow from pump 10 by causing valve 74 to be actuated to a position preventing all flow through housing 36.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A reduced size implantable pump for dispensing an active substance at one or varying flow rates to a patient comprising:
a pump housing defining an interior and having a top surface and a bottom surface;
a valve disposed within the interior, the valve being capable of moving in a direction extending between the top and bottom surfaces;
a propellant envelope disposed within the interior, the propellant envelope defining an active substance chamber and a propellant chamber; and
a hermetic housing attached to the pump housing, the hermetic housing including a first pressure sensor, a second pressure sensor, and an actuator capable of moving the valve.

2. The reduced size implantable pump of claim 1, wherein the pump housing includes a middle part between top and bottom parts.

3. The reduced size implantable pump of claim 2, wherein the valve is disposed within an aperture formed in the middle part.

4. The reduced size implantable pump of claim 2, wherein the hermetic housing is disposed between the bottom and middle parts.

5. The reduced size implantable pump of claim 1, further comprising a catheter fluidly connected with the active substance chamber.

6. The reduced size implantable pump of claim 5, further comprising a resistor capillary fluidly connected with the active substance chamber.

7. The reduced size implantable pump of claim 6, wherein the first pressure sensor measures the pressure of fluid within the active substance chamber, and fluid dispelled from the active substance chamber flows through the resistor capillary, into contact with the second pressure sensor, through the valve, and through the catheter.

8. The reduced size implantable pump of claim 7, wherein pressure information obtained by the first and second pressure sensors is used to determine whether the actuator should actuate the valve.

9. The reduced size implantable pump of claim 8, wherein the hermetic housing further includes a circuit board, motor and battery.

10. The reduced size implantable pump of claim 1, further comprising first and second propellant envelopes.

11. The reduced size implantable pump of claim 10, wherein the first and second propellant envelopes each include a first and second membranes.

12. The reduced size implantable pump of claim 11, wherein each propellant chamber includes a propellant that expands under normal body temperature to act upon the flexible membranes.

13. The reduced size implantable pump of claim 1, wherein the propellant envelope is substantially c-shaped.

14. The reduced size implantable pump of claim 13, further comprising a replenishment port at least partially surrounded by the propellant envelope.

15. A reduced size implantable pump for dispensing an active substance at one or varying flow rates to a patient comprising:
a pump housing defining an interior and having a top surface and a bottom surface;
a valve disposed within the interior, the valve being capable of moving in a direction extending between the top and bottom surfaces;
a first propellant envelope disposed within the interior, the first propellant envelope being c-shaped;
a second propellant envelope disposed within the interior, the second propellant envelope being c-shaped.

16. The reduced size implantable pump of claim 15, further comprising a hermetic housing attached to the pump housing, the hermetic housing including a first pressure sensor, a second pressure sensor, and an actuator capable of moving the valve.

17. The reduced size implantable pump of claim 16, wherein the pump housing includes a middle part between top and bottom parts, the first propellant envelope and a top surface of the middle part defining a first active substance chamber, and the second propellant envelope and a bottom surface of the middle part defining a second active substance chamber.

18. The reduced size implantable pump of claim 17, wherein the valve is disposed within an aperture formed in the middle part.

19. The reduced size implantable pump of claim 18, wherein the hermetic housing is disposed between the bottom and middle parts.

20. The reduced size implantable pump of claim 19, further comprising a catheter fluidly connected with the first and second active substance chambers.

21. The reduced size implantable pump of claim 20, further comprising a resistor capillary fluidly connected with the first and second active substance chambers.

22. The reduced size implantable pump of claim 21, wherein the hermetic housing further includes a circuit board, motor and battery.

23. The reduced size implantable pump of claim 15, wherein the first and second propellant envelopes each include first and second membranes.

24. The reduced size implantable pump of claim 15, further comprising a replenishment port at least partially surrounded by the propellant envelope.
